(19) United States
(12) Patent Application Publication
(10) Pub. No.: US 2025/0034628 A1
(43) Pub. Date: Jan. 30, 2025

(54) NUCLEIC ACID HYBRIDIZATION METHODS

(71) Applicant: Element Biosciences, Inc., San Diego, CA (US)

(72) Inventors: Sinan ARSLAN, San Diego, CA (US); Molly HE, San Diego, CA (US); Michael PREVITE, San Diego, CA (US)

(21) Appl. No.: 18/426,101

(22) Filed: Jan. 29, 2024

Related U.S. Application Data
(63) Continuation of application No. 18/332,469, filed on Jun. 9, 2023, now abandoned.
Application No. 18/332,469 is a continuation of application No. 18/048,552, filed on Oct. 21, 2022, now abandoned.
Application No. 18/048,552 is a continuation of application No. 17/695,494, filed on Mar. 15, 2022, now abandoned.
Application No. 17/695,494 is a continuation of application No. 17/392,655, filed on Aug. 3, 2021, now abandoned.
Application No. 17/392,655 is a continuation of application No. 17/129,106, filed on Dec. 21, 2020, now abandoned.
Application No. 17/129,106 is a continuation of application No. 16/543,351, filed on Aug. 16, 2019, now abandoned.
(60) Provisional application No. 62/841,541, filed on May 1, 2019.

(51) Int. Cl. C12Q 1/6834; B01J 19/00; C12Q 1/6806; C12Q 1/6832

(52) U.S. Cl. CPC C12Q 1/6834; B01J 19/0046; C12Q 1/6806; C12Q 1/6832; B01J 2219/00675

(57) ABSTRACT

Nucleic acid hybridization buffer formulations and uses thereof are described that yield improvements in hybridization specificity, rate, and efficiency. The buffer formulation composition includes a target nucleic acid; at least one organic solvent having a dielectric constant in the range of no greater than 115; and a pH buffer system, wherein the target nucleic acid is attached to the surface via hybridization to a surface bound nucleic acid tethered to the surface, and wherein the hybridization of the target nucleic acid and surface bound nucleic acid has a high stringency and annealing rate.

### CROSS-REFERENCE

This application is a continuation of U.S. application Ser. No. 18/332,469, filed Jun. 9, 2023, which is a continuation of U.S. application Ser. No. 18/048,552, filed Oct. 21, 2022, now abandoned, which is a continuation of U.S. application Ser. No. 17/695,494, filed on Mar. 15, 2022, now abandoned, which is a continuation of U.S. application Ser. No. 17/392,655, filed Aug. 3, 2021, now abandoned, which is a continuation of U.S. application Ser. No. 17/129,106, filed Dec. 21, 2020, now abandoned, which is a continuation of U.S. application Ser. No. 16/543,351, filed on Aug. 16, 2019, now abandoned, which claims the benefit of U.S. Provisional Application No. 62/841,541, filed May 1, 2019, each of which is hereby incorporated by reference in its entirety.

### BACKGROUND

This disclosure herein relates to the field of molecular biology, such as compositions, methods, and systems for nucleic acid hybridization. In particular, it relates to hybridization compositions and methods for nucleic acid that is attached to a surface.

Nucleic acid hybridization protocols constitute an important part of many different nucleic acid amplification and analysis techniques. The limited specificity and reaction rates achieved through the use of existing nucleic acid hybridization protocols can have detrimental effects on the throughput and accuracy of downstream nucleic acid analysis methods. Methods of stringency control often involve conditions causing a significant decrease in the number of hybridized complexes. Therefore, there is a need for an improved method to achieve a high stringency of hybridization during the sequencing analysis.

### SUMMARY

Provided herein are compositions, methods, systems and kits for nucleic acid hybridization prior to or during an amplification process. The compositions and methods disclosed herein allow for high stringency, speed, and efficacy of nucleic acid hybridization and increase the efficiency of the subsequent amplification and sequencing steps. The methods of hybridization or attaching the nucleic acid to a surface includes providing at least one surface bound nucleic acid attached to a surface; and contacting the surface bound nucleic acid to a target nucleic acid in a hybridizing composition, wherein the hybridizing composition comprises: at least one organic solvent, and a pH buffer. The hybridization compositions can include at least one nucleic acid attached to a surface through covalent or noncovalent bond; at least one organic solvent; and a pH buffer system, and the surface has a water contact angle of less than 45 degrees.

The organic solvent can have a dielectric constant of no greater than 115. In some instances, the organic solvent can be a polar aprotic solvent (e.g., formamide). In some instances, the organic solvent can be a solvent having a dielectric constant of no greater than 40 (e.g., acetonitrile or alcohol).

Provided herein includes a method of hybridization, the method comprising: (a) providing at least one surface bound nucleic acid attached to a surface; and (b) contacting the surface bound nucleic acid to a target nucleic acid in a hybridizing composition, wherein the hybridizing composition comprises at least an organic solvent, and a pH buffer; wherein the surface exhibits a level of non-specific cyanine dye 3 (Cy3) dye absorption of less than about 0.25 molecules/μm², and wherein no more than 5% of the target nucleic acid is associated with the surface without hybridizing to the surface bound nucleic acid.

Provided herein includes a method to attach a target nucleic acid to a surface, the method comprising: (a) providing at least one surface bound nucleic acid, wherein the surface bound nucleic acid is tethered to the surface; and (b) contacting the target nucleic acid to the surface bound nucleic acid in the presence of a hybridizing composition, wherein the hybridizing composition comprises: at least one organic solvent, and a pH buffer; wherein the surface exhibits a level of non-specific Cy3 dye absorption of less than about 0.25 molecules/μm², and wherein no more than 5% of the target nucleic acid is associated with the surface without hybridizing to the surface bound nucleic acid. Provided herein includes a method to sequence a target nucleic acid, the method comprising: (a) contacting a target nucleic acid to a nucleic acid tethered in the presence of a hybridizing composition, wherein the hybridizing composition comprises at least one organic solvent and a pH buffer; (b) amplifying the target nucleic acid to form a plurality of clonally-amplified clusters of nucleic acid, and (c) determining the sequence of the target nucleic acid, wherein a fluorescence image of the surface having the plurality of clonally-amplified clusters of nucleic acid exhibits a contrast-to-noise ratio (CNR) of at least 20 using a fluorescence imaging system under non-signal saturating conditions.

Provided herein includes a composition to attach a target nucleic acid to a surface, comprising: a target nucleic acid; at least one organic solvent; and a pH buffer system, wherein the target nucleic acid is attached to the surface via hybridization to a surface bound nucleic acid tethered to the surface, and wherein the hybridization of the target nucleic acid and surface bound nucleic acid has a stringency of at least 70%, at least 80%, or at least 90% and wherein no more than 5% of the target nucleic acid is bound to the surface without hybridizing to the surface bound nucleic acid.

Provided herein also includes compositions and methods to enhance nucleic acid hybridization. The methods to enhance nucleic acid hybridization at a surface can include providing at least one surface bound nucleic acid, wherein the surface bound nucleic acid is bound to the surface through covalent or noncovalent bond; and contacting a target nucleic acid to the surface bound nucleic acid under conditions of stringency that presents the target nucleic acid from hybridizing to a non-complementary nucleic acid. The compositions to enhance nucleic acid hybridization at a surface can include at least one nucleic acid attached to a surface through covalent or noncovalent bond; at least one organic solvent; and a pH buffer system, wherein the nucleic acid attached to the surface hybridizes with a target nucleic acid with a stringency of at least 80%, where stringency is defined as the percentage of bases, within a hybridization region or within a subset of sequence undergoing hybridization, which must be complementary through standard Watson-Crick base pairing (e.g., for a hybridization stringency of 80%, 80% of the bases in the hybridized segment must participate in standard base pairs). As used herein, the hybridization region may comprise the region of sequence defined by a surface-bound nucleic acid and/or a nucleic acid identified as a “probe.” Some Additional embodiments include method for a method to sequence a target nucleic acid, the method comprising: contacting a target nucleic acid to a nucleic acid attached to a surface through covalent or noncovalent bond under conditions of stringency that presents the target nucleic acid from hybridizing to a non-complementary nucleic acid; amplifying the target nucleic acid, and determining the sequence of the target nucleic acid

Some embodiments relate to a method to attach a target nucleic acid to a surface, the method comprising: providing at least one surface bound nucleic acid, wherein the surface bound nucleic acid is tethered to the surface; and contacting the target nucleic acid to the surface bound nucleic acid in the presence of a hybridizing composition, wherein the hybridizing composition comprises: at least one organic solvent, and a pH buffer; wherein the surface exhibits a level of non-specific Cy3 dye absorption of less than about 0.25 molecules/μm2, and wherein no more than 5% of the target nucleic acid is associated with the surface without hybridizing to the surface bound nucleic acid.

Some embodiments relate to a method to sequence a target nucleic acid, the method comprising: contacting a target nucleic acid to a nucleic acid tethered in the presence of a hybridizing composition, wherein the hybridizing composition comprises at least one organic solvent and a pH buffer; amplifying the target nucleic acid to form a plurality of clonally-amplified clusters of nucleic acid, and determining the sequence of the target nucleic acid, wherein a fluorescence image of the surface having the plurality of clonally-amplified clusters of nucleic acid exhibits a contrast-to-noise ratio (CNR) of at least 20 using a fluorescence imaging system under non-signal saturating conditions. In some embodiments, the contrast to noise ratio (CNR) is at least 50.

Some embodiments relate to a composition to attach a target nucleic acid to a surface, comprising: a target nucleic acid; at least one organic solvent; and a pH buffer system, wherein the target nucleic acid is attached to the surface via hybridization to a surface bound nucleic acid tethered to the surface, and wherein the hybridization of the target nucleic acid and surface bound nucleic acid has a stringency of at least 70%, at least 80%, or at least 90% and wherein no more than 5% of the target nucleic acid is bound to the surface without hybridizing to the surface bound nucleic acid.

Some embodiments relate to a microfluidic system, comprising the composition described herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some novel features of the methods and compositions disclosed herein are set forth in the present disclosure. A better understanding of the features and advantages of the methods and compositions disclosed herein will be obtained by reference to the following detailed description that sets forth illustrative embodiments, in which the principles of the disclosed compositions and methods are utilized, and the accompanying drawings of which:

FIGS. 1A-1B provide non-limiting examples of image data that demonstrate the improvements in hybridization stringency, speed, and efficacy that may be achieved through the reformulation of the hybridization buffer used for solid-phase nucleic acid amplification, as described herein. FIG. 1A provides examples of image data for two different hybridization buffer formulations and protocols.

FIG. 1B provides an example of the corresponding image data obtained using a standard hybridization buffer and protocol.

FIG. 2 illustrates a workflow for nucleic acid sequencing using the disclosed hybridization methods on low binding surfaces, and non-limiting examples of the processing times that may be achieved.

FIG. 3 shows the surface template hybridization images (NASA results at 100 pM) of the samples corresponding to the compositions used for hybridization.

FIG. 4 shows a table with hybridization DOE spot counts.

FIG. 5 shows the post NASA amplification PCR images of the samples.

### DETAILED DESCRIPTION

Disclosed herein are improved methods, compositions, and systems for nucleic acid hybridization that provide faster reaction kinetics and increased reaction specificity using reduced quantities of input nucleic acid, particularly when used in combination with low nonspecific binding surfaces. Conventional hybridization methods are complex and time consuming. For example, such conventional methods can lack specificity and/or efficiency, and can require high temperature incubations, large sample inputs, and/or long incubation times. The methods described herein provide unexpectedly faster annealing, decreased sample input requirements, high efficiency and specificity, and significantly shortener hybridization times. In addition, the annealing can be performed at isothermal conditions and eliminate the cooling step for annealing. Hybridization buffer formulations are described which, in combination with the disclosed low-binding supports, provide for improved hybridization rates, hybridization specificity (or stringency), and hybridization efficiency (or yield). As used herein, hybridization specificity is a measure of the ability of tethered adapter sequences, primer sequences, or oligonucleotide sequences in general to correctly hybridize only to completely complementary sequences, while hybridization efficiency is a measure of the percentage of total available tethered adapter sequences, primer sequences, or oligonucleotide sequences in general that are hybridized to complementary sequences.

The annealing method described herein in combination with a low nonspecific binding surface and amplification protocols can lead to one or more of: (i) improved hybridization rates, (ii) hybridization specificity (or stringency), and (iii) hybridization efficiency (or yield), (iv) reduced requirements for the amount of starting material necessary, (v) lowered temperature requirements for isothermal or thermal ramping amplification protocols, (vi) increased annealing rates, (vii) increased annealing specificity (that is, more selective annealing of the single-stranded template molecules while decreasing annealing of nontarget nucleic acid molecules), and (ix) yield a low percentage of the target nucleic acid being associated with the surface without hybridizing to the surface bound nucleic acid.

Improvements in hybridization reaction kinetics and specificity may be achieved through the use of hybridization formulations that comprise: at least one organic solvent having a dielectric constant in the range of no greater than 115, and a pH buffer. In addition, the formulation can comprise molecular crowding/volume exclusion agents; additives that impact DNA melting temperatures, additives that impact DNA hydration, and or any combination thereof. Various aspects of the disclosed nucleic acid hybridization methods may be applied not only to solution-phase or solid-phase nucleic acid hybridization, but also to any other type of nucleic acid amplification and/or analysis applications (e.g., nucleic acid sequencing) known to those of skill in the art. It shall be understood that different aspects of the disclosed methods, devices, and systems can be appreciated individually, collectively, or in combination with each other.

Without intending to be bound by any particular theory, it has been noted that the hybridization reaction or annealing interaction between nucleic acids in the solution and nucleic acids attached to a hydrophilic surface can be related to several factors including the availability of hydrogen bonding partners in the solution and the polarity of the solution. When the solution contains a protic solvent that helps provide sufficient hydrogen bonding partners, of sufficient size and distribution, the hydrogen bonding interactions between the exposed hydrogen bond donors and acceptors along the nucleic acid backbone and/or any exposed sidechain moieties provides a favorable environment for the nucleic acid to stay in solution rather than binding to the hydrophilic surface.

In addition, nucleic acids preferentially inhabit bulk solution where possible in order to take advantage of the additional entropic stabilization presented by the ability to access dynamic states in three, rather than two, dimensions such as would be available on a hydrophilic surface. It is thus understood that, at equilibrium, in a system comprising a nucleic acid, a solution, and a hydrophilic surface, a nucleic acid molecule will be preferentially stabilized in solution rather than in a surface-bound state when the solvent is aqueous. However, by using an aprotic solvent such as formamide and reducing the proportion of solvent molecules capable of satisfying the hydrogen bonding requirements of the nucleic acid chain, it is possible to create an entropic penalty in the bulk solution which will drive the system toward stabilization by depositing the nucleic acid on the surface (i.e., the entropic penalty caused by ordering the bulk solution to accommodate the unbonded hydrogen bonding elements in the nucleic acid becomes greater than the entropic penalty caused by loss of the third dimension of dynamic freedom when the polymer is adsorbed to the surface). Furthermore, introduction of an aprotic organic solvent into the solution may help drive down the entropy and in turn provides a more favorable environment for the nucleic acid to bind to the hydrophilic surface. For example, addition of an aprotic r solvent acetonitrile helps to drive the nucleic acid in the solution towards a surface bound state.

It has been noted that a high proportion of aprotic solvent and/or aprotic solvent in the solution can result in precipitation of the nucleic acid from the solution. Thus, addition of aprotic and aprotic solvents within the concentration ranges described herein, to solutions comprising nucleic acids, can cause the nucleic acids to selectively associate with hydrophilic surfaces while remaining substantially solvated. These same thermodynamic parameters govern a number of interactions between polymers and biomolecules, as well as polymer/surface and biomolecule/surface interactions, and thus tuning the polarity and/or the hydrogen bonding potential of the solvent as disclosed herein may represent a method of tuning such interactions in applications beyond nucleic acid/surface interactions.

It has been determined that components capable of modulating interactions of nucleic acids with the bulk solution, such as, for example, crowding agents; or components capable of modulating the dynamics of the polymer itself such as, for example, “relaxing” agents, divalent cations, or intercalating agents, may also modulate the interactions of nucleic acids with surfaces in the presence of partially aprotic bulk solvents. Further, providing such agents in combination with buffers containing some fraction of aprotic or non-hydrogen-bonding components can in some cases provide superior control over the interaction of nucleic acid molecules with hydrophilic surfaces.

The annealing methods described herein can be used in combination with a passivated low binding surface. As a result of the surface passivation techniques disclosed herein, proteins, nucleic acids, and other biomolecules do not “stick” to the substrates, that is, they exhibit low nonspecific binding (NSB). Conventional hybridization formulation would not work well with the passivated NSB surface. Hydrophilic surface that have been passivated to achieve ultra-low NSB for proteins and nucleic acids require novel hybridization performance. The annealing methods and formulations described herein unexpectedly work well with a low NSB surface. More specifically, it was unexpected that the annealing methods and formulations described herein work well with the surface having a level of non-specific Cy3 dye absorption of less than about 0.25 molecules/μm² to achieve a high annealing efficiency of no more than 5% of the target nucleic acid is associated with the surface without hybridizing to the surface bound nucleic acid.

### ABBREVIATION

Dimethyl sulfoxide (DMSO),
Dimethyl formamide (DMF),
3-(N-morpholino) propanesulfonic acid (MOPS),
Acetonitrile (ACN)
2-(N-morpholino) ethanesulfonic acid (MES)
saline-sodium citrate (SSC)
Formamide (Form.)
Tris(hydroxymethyl)aminomethane (Tris)

Nucleic acid hybridization kinetics and specificity: Improved DNA hybridization conditions have widely varying applications for nucleic acid assays. DNA hybridization in solution between two ssDNA molecules of complementary sequence is governed by strong non-covalent attraction between base pairs to form hydrogen bonds and create a duplex DNA structure (J. D. Watson, JAMA, 1993, 269, 1966). Solution-based hybridization is the foundation for many solution-based molecular biology and solution-phase DNA manipulation applications, most notably the polymerase chain reaction (PCR) (L. Garibyan and N. Avashia, J. Invest. Dermatol., 2013, 133, e6; Z. Xiao, D. Shangguan, Z. Cao, X. Fang, and W. Tan, 2008, DNA guided drug delivery, Chemistry 14, 1769-75; and F. Wei, C. Chen, L. Zhai, N. Zhang, and X. S. Zhao, 2005, DNA based biosensors, J. Am. Chem. Soc., 127, 5306-5307; and S. Tyagi and F. R. Kramer, Nat. Biotechnol., 1996, 14, 303-308. The diffusion rates in many of these reactions are sufficient to drive efficient hybridization and the formation of a functional double-stranded form, which can be analyzed kinetically as a second order kinetic reaction, whereby the forward reaction of duplex formation is second order and the reverse reaction comprising the dissociation of the duplex structure to form the two single stranded complements (strands A and B) is first order (Han, C., Improvement of the Speed and Sensitivity of DNA Hybridization Using Isotachophoresis, Stanford Thesis. 2015). These reactions may be written as:

$$A + B {{⇄}^{{k}_{on}}}_{{k}_{off}} AB$$

$$\frac{d [ AB ]}{dt} = {k}_{on} [ A ] [ B ] - {k}_{off} [ AB ]$$

Various approaches have been deployed to increase not only the speed of the hybridization reaction but also the reaction specificity in the wake of confounding DNA non-complementary fragments. Such approaches include, but are not limited to, the addition of MgCl₂ and higher salt concentrations, and lower temperatures to accelerate the reactions (H. Kuhn, V. V Demidov, J. M. Coull, M. J. Fiandaca, B. D. Gildea, and M. D. Frank-Kamenetskii, J. Am. Chem. Soc., 2002, 124, 1097-1103; N. A. Straus and T. I. Bonner, Biochim. Biophys. Acta, Nucleic Acids Protein Synth., 1972, 277, 87-95). The trade-off for accelerated reaction rates is often reaction specificity (J. M. S. Bartlett and D. Stirling, PCR protocols, Humana Press, 2003; W. Rychlik, W. J. Spencer, and R. E. Rhoads, Nucleic Acids Res., 1990, 18). Additional methods are sometimes employed that yield potential improvements of reaction specificity through the use of volume exclusion and/or molecular crowding techniques that utilize inert polymers as hybridization buffer additives (R. Wieder and J. G. Wetmur, Biopolymers, 1981, 20, 1537-1547, J. G. Wetmur, Biopolymers, 1975, 14, 2517-2524). In addition, organic solvents have been employed as additives to accelerate hybridization kinetics and maintain reaction specificity (N. Dave and J. Liu, J. Phys. Chem. B, 2010, 114, 15694-15699).

While hybridization improvements in solution may be translated to surface-based hybridization techniques, surface-based hybridization needs have far ranging implications for many critical bioassays, such as gene expression analysis (D. T. Ross, U. Scherf, M. B. Eisen, C. M. Perou, C. Rees, P. Spellman, V. Iyer, S. S. Jeffrey, M. Van de Rijn, M. Waltham, A. Pergamenschikov, J. C. Lee, D. Lashkari, D. Shalon, T. G. Myers, J. N. Weinstein, D. Botstein, and P. O. Brown, Nat. Genet., 2000, 24, 227-235; A. Adomas, G. Heller, A. Olson, J. Osborne, M. Karlsson, J. Nahalkova, L. Van Zyl, R. Sederoff, J. Stenlid, R. Finlay, and F. O. Asiegbu, Tree Physiol., 2008, 28, 885-897; M. Schena, D. Shalon, R. W. Davis, and P. O. Brown, Science, 1995, 270, 467-470), diagnosis of disease (J. Marx, Science, 2000, 289, 1670-1672), genotyping and SNP detection (J. G. Hacia, J. B. Fan, O. Ryder, L. Jin, K. Edgemon, G. Ghandour, R. A. Mayer, B. Sun, L. Hsie, C. M. Robbins, L. C. Brody, D. Wang, E. S. Lander, R. Lipshutz, S. P. Fodor, and F. S. Collins, Nat. Genet., 1999, 22, 164-167), rapid pathogen nucleic acid based pathogen screening, next generation sequencing (NGS) and a host of other genomics based applications (M. J. Heller, Annu. Rev. Biomed. Eng., 2002, 4, 129-53). The common necessity of all of these reactions is high reaction specificity in a highly multiplexed solution of target sequences that may range from thousands to billions of different sequences, such that the targets are quickly tethered on a solid surface for subsequent probing and/or amplification to enable DNA (or other nucleic acid) interrogation for applications such as sequencing or array-based analysis. The efficiency of surface-based hybridization reactions were found to be much less than that of in solution reactions, e.g., about an order of magnitude less efficient. A great deal of work has been done in past attempts to create a hybridization method for solid surface s that provides high specificity and accelerated hybridization reaction rates (D. Y. Zhang, S. X. Chen, and P. Yin, Nat. Chem., 2012, 4, 208-14).

In this disclosure, novel combinations of approaches gleaned from studies of surface- and solution-based hybridization as outlined above, as well as from other fields of study that include DNA hydration and quadruplex studies (Petracone, et al., Methods, 2012; Hong, et al., Biochemistry, 2004), are described which lead to substantial improvements in hybridization kinetics and specificity. The disclosed hybridization formulations provide for highly specific (>2 orders of magnitude improvement over traditional approaches) and accelerated hybridization (>1-2 orders of magnitude improvement over traditional approaches) when used with low non-specific binding solid surface for applications such as next generation sequencing (NGS) and other bioassays that require highly specific nucleic acid hybridization in a multiplexed pool comprised of large number of target sequences.

Rapid, specific DNA hybridization formulations for use with low non-specific binding solid surfaces, such as silicon dioxide coated with low binding polymers such as PEG (or other low binding substrates as outlined in co-pending U.S. Provisional Patent Application No. 62/767,343) for sequencing, genotyping, or sequencing related technologies may be achieved using any or a combination of the following hybridization buffer components.

The nucleic acid hybridization method and compositions described herein are useful for annealing the target nucleic acid to the nucleic acid tethered to the surface with high stringency and speed. The methods described here utilize the hybridization composition and the low nonspecific binding surface to achieve an improved hybridization and prepare the target nucleic acid for the amplification step. The combination of the hybridizing buffer and the surface confer one or more of the following advantages in a sequencing process: (i) decreased fluidic wash times (due to reduced non-specific binding, and thus faster sequencing cycle times), (ii) decreased imaging times (and thus faster turnaround times for assay readout and sequencing cycles), (iii) decreased overall work flow time requirements (due to decreased cycle times), (iv) decreased detection instrumentation costs (due to the improvements in CNR), (v) improved readout (base-calling) accuracy (due to improvements in CNR), (vi) improved reagent stability and decreased reagent usage requirements (and thus reduced reagents costs), and (vii) fewer run-time failures due to nucleic acid amplification failures.

The surface bound nucleic acid can be attached to the surface vis a number of suitable options. In some instance, the nucleic acids is attached to the surface through covalent bond. In some embodiments, the nucleic acids is attached to the surface through noncovalent bond. nucleic acids is attached to the surface through biointeraction such as biotin-streptavidin interactions (or variations thereof), his tag-Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The nucleic acid hybridization method and compositions described herein are useful for preparing a hybridization library prior to or during the amplification step. The hybridization composition described herein can include at least one nucleic acid attached to a surface through covalent or noncovalent bond; at least one polar and/or polar aprotic solvent; and a pH buffer system. The method of hybridizing a target nucleic acid to a surface bound nucleic acid can include contacting the target nucleic acid to the hybridizing composition described herein. The combination of the agents in the hybridizing composition allows for a high stringency hybridization process and also enhance the efficiency of the subsequent amplification process.

Definitions: Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the term ‘about’ a number refers to that number plus or minus 10% of that number. The term ‘about’ when used in the context of a range refers to that range minus 10% of its lowest value and plus 10% of its greatest value.

As used herein, the terms “DNA hybridization” and “nucleic acid hybridization” are used interchangeably, and are intended to cover any type of nucleic acid hybridization, e.g., DNA hybridization, RNA hybridization, etc., unless otherwise specified.

Organic Solvent: An organic solvent is a solvent or solvent system comprising carbon-based or carbon-containing substance capable of dissolving or dispersing other substances. An organic solvent may be miscible or immiscible with water.

Polar Solvent: A polar solvent as included in the hybridization composition described herein is a solvent or solvent system comprising one or more molecules characterized by the presence of a permanent dipole moment, i.e., a molecule having a spatially unequal distribution of charge density. A polar solvent may be characterized by a dielectric constant of 20, 25, 30, 35, 40, 45, 50, 55, 60 or higher or by a value or a range of values incorporating any of the aforementioned values. For example, a polar solvent may have a dielectric constant of higher than 100, higher than 110, higher than 111, or higher than 115. A polar solvent as described herein may comprise a polar aprotic solvent. A polar aprotic solvent as described herein may further contain no ionizable hydrogen in the molecule. In addition, polar solvents or polar aprotic solvents may be preferably substituted in the context of the presently disclosed compositions with a strong polarizing functional groups such as nitrile, carbonyl, thiol, lactone, sulfone, sulfite, and carbonate groups so that the underlying solvent molecules have a dipole moment. Polar solvents and polar aprotic solvents can be present in both aliphatic and aromatic or cyclic form. In some embodiments, the polar solvent is acetonitrile.

The organic solvent described herein can have a dielectric constant that is the same as or close to acetonitrile. The dielectric constant of the organic solvent can be in the range of about 20-60, about 25-55, about 25-50, about 25-45, about 25-40, about 30-50, about 30-45, or about 30-40. The dielectric constant of the organic solvent can be greater than 20, 25, 30, 35, or 40. The dielectric constant of the organic solvent can be lower than 30, 40, 45, 50, 55, or 60. The dielectric constant of the organic solvent can be about 35, 36, 37, 38, or 39.

Dielectric constant may be measured using a test capacitor according to methods known in the art. Representative polar aprotic solvents having a dielectric constant between 30 and 120 may include any such solvent as is known in the art or disclosed elsewhere herein. Such solvents may particularly include, but are not limited to, acetonitrile, diethylene glycol, N,N-dimethylacetamide, dimethyl formamide, dimethyl sulfoxide, ethylene glycol, formamide, hexamethylphosphoramide, glycerin, methanol, N-methyl-2-pyrrolidinone, nitrobenzene, or nitromethane.

The organic solvent described herein can have a polarity index that is the same as or close to acetonitrile. The polarity index of the organic solvent can be in the range of about 2-9, 2-8, 2-7, 2-6, 3-9, 3-8, 3-7, 3-6, 4-9, 4-8, 4-7, or 4-6. The polarity index of the organic solvent can be greater than about 2, 3, 4, 4.5, 5, 5.5, or 6. The polarity index of the organic solvent can be lower than about 4.5, 5, 5.5, 6, 6.5, 7, 7.5, 8, 8.5, 9, or 10. The polarity index of the organic solvent can be about 5.5, 5.6, 5.7, or 5.8.

The Snyder Polarity Index may be calculated according to the methods disclosed in Snyder, L. R., Journal of Chromatography A, 92 (2): 223-30 (1974), which is incorporated by reference herein in it its entirety. Representative polar aprotic solvents having a Snyder polarity index between 6.2 and 7.3 may include any such solvent as is known in the art or disclosed elsewhere herein. Such solvents may particularly include, but are not limited to, acetonitrile, dimethyl acetamide, dimethyl formamide, N-methyl pyrrolidone, N,N-dimethyl sulfoxide, methanol, or formamide.

Relative polarity may be determined according to the methods given in Reichardt, C., Solvents and Solvent Effects in Organic Chemistry, 3rd ed., 2003, which is incorporated herein by reference in its entirety, and especially with respect to its disclosure of polarities and methods of determining or assessing the same for solvents and solvent molecules. Representative polar aprotic solvents having a relative polarity between 0.44 and 0.82 may include any such solvent as is known in the art or disclosed elsewhere herein. Such solvents may particularly include, but are not limited to, dimethylsulfoxide, acetonitrile, 3-pentanol, 2-pentanol, 2-butanol, Cyclohexanol, 1-octanol, 2-propanol, 1-heptanol, i-butanol, 1-hexanol, 1-pentanol, acetyl acetone, ethyl acetoacetate, 1-butanol, benzyl alcohol, 1-propanol, 2-aminoethanol, Ethanol, diethylene glycol, methanol, ethylene glycol, glycerin, or formamide.

The Solvent Polarity (Er (30)) may be calculated according to the methods disclosed in Reichardt, C., Molecular Interactions, Volume 3, Ratajczak, H. and Orville, W. J., Eds (1982), which is incorporated by reference herein in it its entirety.

Some examples of organic solvent include but are not limited to acetonitrile, dimethylformamide (DMF), dimethylsulfoxide (DMSO), acetanilide, N-acetyl pyrrolidone, 4-amino pyridine, benzamide, benzimidazole, 1,2,3-benzotriazole, butadienedioxide, 2,3-butylene carbonate, γ-butyrolactone, caprolactone (epsilon), chloro maleic anhydride, 2-chlorocyclohexanone, chloroethylene carbonate, chloronitromethane, citraconic anhydride, crotonlactone, 5-cyano-2-thiouracil, cyclopropylnitrile, dimethyl sulfate, dimethyl sulfone, 1,3-dimethyl-5-tetrazole, 1,5-dimethyl tetrazole, 1,2-dinitrobenzene, 2,4-dinitrotoluene, dipheynyl sulfone, 1,2-dinitrobenzene, 2,4-dinitrotoluene, dipheynyl sulfone, epsilon-caprolactam, ethanesulfonylchloride, ethyl ethyl phosphinate, N-ethyl tetrazole, ethylene carbonate, ethylene trithiocarbonate, ethylene glycol sulfate, ethylene glycol sulfite, furfural, 2-furonitrile, 2-imidazole, isatin, isoxazole, malononitrile, 4-methoxy benzonitrile, 1-methoxy-2-nitrobenzene, methyl alpha bromo tetronate, 1-methyl imidazole, N-methyl imidazole, 3-methyl isoxazole, N-methyl morpholine-N-oxide, methyl phenyl sulfone, N-methyl pyrrolidinone, methyl sulfolane, methyl-4-toluenesulfonate, 3-nitroaniline, nitrobenzimidazole, 2-nitrofuran, 1-nitroso-2-pyrolidinone, 2-nitrothiophene, 2-oxazolidinone, 9,10-phenanthrenequinone, N-phenyl sydnone, phthalic anhydride, picolinonitrile (2-cyanopyridine), 1,3-propane sultone, β-propiolactone, propylene carbonate, 4H-pyran-4-thione, 4H-pyran-4-one (γ-pyrone), pyridazine, 2-pyrrolidone, saccharin, succinonitrile, sulfanilamide, sulfolane, 2,2,6,6-tetrachlorocyclohexanone, tetrahydrothiapyran oxide, tetramethylene sulfone (sulfolane), thiazole, 2-thiouracil, 3,3,3-trichloro propene, 1,1,2-trichloro propene, 1,2,3-trichloro propene, trimethylene sulfide-dioxide, and trimethylene sulfite.

Representative polar aprotic solvents having a solvent polarity between 44 and 60 may include any such solvent as is known in the art or disclosed elsewhere herein. Such solvents may particularly include, but are not limited to, dimethyl sulfoxide, 2-methoxycarbonylphenol, triethyl phosphite, 3-pentanol, acetonitrile, nitromethane, cyclohexanol, 2-pentanol, 4-methyl-1,3, dioxolan-2-one, propylene carbonate, acrylonitrile, 1-phenylethanol, 1-dodecanol, 2-butanol, 2-methylcyclohexanol, 2,6, dimethylphenol, 2,6-xylenol, 1-decanol, cyclopentanol, dimethyl sulfone, 1-octanoldiethylene glycol mono n-butyl ether, butyl digol, 1-heptanol, 3-phenyl-1-propanol, 1,3-dioxolane-2-one, ethylene carbonate, 1-hexanol, 4-chlorobutyronitrile, 5-methyl-2-isopropylphenol, thymol, 3,5,5-trimethyl-1-hexanol, 3-methyl-1-butanol, isoamyl alcohol, 2-methyl-1-propanol, isobutyl alcohol, 2-(tert-butyl) phenol, 1-pentanol, 2-phenylethanol, 2-methylpentane-2,4-diol, dipropylene glycol, 2-isopropylphenol, 2-n-butoxyethanol, ethylene glycol mono-n-butyl ether, 1-butanol, 2-hydroxymethyl-tetrahydrofuran, tetrahydrofurfuryl alcohol, 2-hydroxymethylfuran, furfuryl alcohol, 1-propanol, 2,4-dimethylphenol, 2,4-xylenol, benzyl alcohol, 2-ethoxyphenol, 2-ethoxyethanol, 1,5-pentanediol, 1-bromo-2-propanol, 2-methyl-5-isopropylphenol, carvacrol, 2-aminoethanol, ethanol, n-methylacetamide, 3-chloropropionitrile, 2-propen-1-ol, allyl alcohol, 2-methoxyethanol, 2-methylphenol, o-cresol, 1,3-butanediol, 2-propyn-1-ol, propargyl alcohol, 3-methylphenol, m-cresol, triethylene glycol, diethylene glycol, n-methylformamide, 1,2-propanediol, 1,3-propanediol, 2-chlorophenol, methanol, 1,2-ethanediol, glycol, formamide, 2,2,2-trichloroethanol, 1,2,3-propanetriol, glycerol, 2,2,3,3-tetrafluoro-1-propanol, 2,2,2-trifluoroethanol, 4-n-butylphenol, 4-methylphenol, or p-cresol.

Representative polar aprotic solvents having a dielectric constant in the range of about 30-115 may include any such solvent as is known in the art or disclosed elsewhere herein. Such solvents may particularly include, but are not limited to, dimethyl sulfoxide, 2-methoxycarbonylphenol, triethyl phosphite, 3-pentanol, acetonitrile, nitromethane, cyclohexanol, 2-pentanol, 4-methyl-1,3, dioxolan-2-one, propylene carbonate, acrylonitrile, 1-phenylethanol, 1-dodecanol, 2-butanol, 2-methylcyclohexanol, 2,6,dimethylphenol, 2,6-xylenol, 1-decanol, cyclopentanol, dimethyl sulfone, 1-octanoldiethylene glycol mono n-butyl ether, butyl digol, 1-heptanol, 3-phenyl-1-propanol, 1,3-dioxolane-2-one, ethylene carbonate, 1-hexanol, 4-chlorobutyronitrile, 5-methyl-2-isopropylphenol, thymol, 3,5,5-trimethyl-1-hexanol, 3-methyl-1-butanol, isoamyl alcohol, 2-methyl-1-propanol, isobutyl alcohol, 2-(tert-butyl) phenol, 1-pentanol, 2-phenylethanol, 2-methylpentane-2,4-diol, dipropylene glycol, 2-isopropylphenol, 2-n-butoxyethanol, ethylene glycol mono-n-butyl ether, 1-butanol, 2-hydroxymethyl-tetrahydrofuran, tetrahydrofurfuryl alcohol, 2-hydroxymethylfuran, furfuryl alcohol, 1-propanol, 2,4-dimethylphenol, 2,4-xylenol, benzyl alcohol, 2-ethoxyphenol, 2-ethoxyethanol, 1,5-pentanediol, 1-bromo-2-propanol, 2-methyl-5-isopropylphenol, carvacrol, 2-aminoethanol, ethanol, n-methylacetamide, 3-chloropropionitrile, 2-propen-1-ol, allyl alcohol, 2-methoxyethanol, 2-methylphenol, o-cresol, 1,3-butanediol, 2-propyn-1-ol, propargyl alcohol, 3-methylphenol, m-cresol, triethylene glycol, diethylene glycol, n-methylformamide, 1,2-propanediol, 1,3-propanediol, 2-chlorophenol, methanol, 1,2-ethanediol, glycol, formamide, 2,2,2-trichloroethanol, 1,2,3-propanetriol, glycerol, 2,2,3,3-tetrafluoro-1-propanol, 2,2,2-trifluoroethanol, 4-n-butylphenol, 4-methylphenol, or p-cresol.

Organic solvent addition: In some instances, the disclosed hybridization buffer formulations may include the addition of an organic solvent. Examples of suitable solvents include, but are not limited to, acetonitrile, ethanol, DMF, and methanol, or any combination thereof at varying percentages (typically >5%). In some instances, the percentage of organic solvent (by volume) included in the hybridization buffer may range from about 1% to about 20%. In some instances, the percentage by volume of organic solvent may be at least 1%, at least 2%, at least 3%, at least 4%, at least 5%, at least 6%, at least 7%, at least 8%, at least 9%, at least 10%, at least 15%, or at least 20%. In some instances, the percentage by volume of organic solvent may be at most 20%, at most 15%, at most 10%, at most 9%, at most 8%, at most 7%, at most 6%, at most 5%, at most 4%, at most 3%, at most 2%, or at most 1%. Any of the lower and upper values described in this paragraph may be combined to form a range included within the present disclosure, for example, the percentage by volume of organic solvent may range from about 4% to about 15%. Those of skill in the art will recognize that the percentage by volume of organic solvent may have any value within this range, e.g., about 7.5%.

When the organic solvent comprises a polar aprotic solvent, the amount of the polar aprotic solvent is present in an amount effective to denature a double stranded nucleic acid. In some embodiments, the amount of the polar aprotic solvent is greater than about 10% by volume based on the total volume of the formulation. The amount of the polar aprotic solvent is about or more than about 5%, 10%, 15%, 20%, 25%, 30%, 35%, 40%, 50%, 60%, 70%, 80%, 90%, or higher, by volume based on the total volume of the formulation. The amount of the polar aprotic solvent is lower than about 15%, 20%, 25%, 30%, 35%, 40%, 50%, 60%, 70%, 80%, 90%, or higher, by volume based on the total volume of the formulation. In some embodiments, the amount of the polar aprotic solvent is in the range of about 10% to 90% by volume based on the total volume of the formulation. In some embodiments, the amount of the polar aprotic solvent is in the range of about 25% to 75% by volume based on the total volume of the formulation. In some embodiments, the amount of the polar aprotic solvent is in the range of about 10% to 95%, 10% to 85%, 20% to 90%, 20% to 80%, 20% to 75%, or 30% to 60% by volume based on the total volume of the formulation. In some embodiments, the polar aprotic solvent is formamide.

When the organic solvent comprises a polar aprotic solvent, the amount of the aprotic solvent is present in an amount effective to denature a double stranded nucleic acid. In some embodiments, the amount of the aprotic solvent is greater than about 10% by volume based on the total volume of the formulation. The amount of the aprotic solvent is about or more than about 5%, 10%, 15%, 20%, 25%, 30%, 35%, 40%, 50%, 60%, 70%, 80%, 90%, or higher, by volume based on the total volume of the formulation. The amount of the aprotic solvent is lower than about 15%, 20%, 25%, 30%, 35%, 40%, 50%, 60%, 70%, 80%, 90%, or higher, by volume based on the total volume of the formulation. In some embodiments, the amount of the aprotic solvent solvent is in the range of about 10% to 90% by volume based on the total volume of the formulation. In some embodiments, the amount of the aprotic solvent solvent is in the range of about 25% to 75% by volume based on the total volume of the formulation. In some embodiments, the amount of the aprotic solvent solvent is in the range of about 10% to 95%, 10% to 85%, 20% to 90%, 20% to 80%, 20% to 75%, or 30% to 60% by volume based on the total volume of the formulation.

Addition of molecular crowding volume exclusion agents: The composition described herein can include one or more crowding agents enhances molecular crowding. The crowding agent can be selected from the group consisting of polyethylene glycol (PEG), dextran, hydroxypropyl methyl cellulose (HPMC), hydroxyethyl methyl cellulose (HEMC), hydroxybutyl methyl cellulose, hydroxypropyl cellulose, methycellulose, and hydroxyl methyl cellulose, and combination thereof. A preferred crowding agent may comprise one or more of polyethylene glycol (PEG), dextran, proteins, such as ovalbumin or hemoglobin, or Ficoll.

A suitable amount of a crowding agent in the composition allows for, enhances, or facilitates molecular crowding. The amount of the crowding agent is about or more than about 1%, 2%, 3%, 5%, 10%, 15%, 20%, 25%, 30%, 35%, 40%, 50%, 60%, or higher, by volume based on the total volume of the formulation. In some cases, the amount of the molecular crowding agent is greater than 5% by volume based on the total volume of the formulation. The amount of the crowding agent is lower than about 3%, 5%, 10%, 12.5%, 15%, 20%, 25%, 30%, 35%, 40%, 50%, 60%, 70%, 80%, 90%, or higher, by volume based on the total volume of the formulation. In some cases, the amount of the molecular crowding agent can be less than 30% by volume based on the total volume of the formulation. In some embodiments, the amount of the organic solvent is in the range of about 25% to 75% by volume based on the total volume of the formulation. In some embodiments, the amount of the organic solvent is in the range of about 1% to 40%, 1% to 35%, 2% to 50%, 2% to 40%, 2% to 35%, 2% to 30%, 2% to 25%, 2% to 20%, 2% to 10%, 5% to 50%, 5% to 40%, 5% to 35%, 5% to 30%, 5% to 25%, 5% to 20%, by volume based on the total volume of the formulation. In some cases, the amount of the molecular crowding agent can be in the range of about 5% to about 20% by volume based on the total volume of the formulation. In some embodiments, the amount of the crowding agent is in the range of about 1% to 30% by volume based on the total volume of the formulation.

One example of the crowding agent in the composition is polyethylene glycol (PEG. In some embodiments, the PEG used can have a molecular weight sufficient to enhance or facilitate molecular crowding. In some embodiments, the PEG used in the composition has a molecular weight in the range of about 5 k-50 k Da. In some embodiments, the PEG used in the composition has a molecular weight in the range of about 10 k-40 k Da. In some embodiments, the PEG used in the composition has a molecular weight in the range of about 10 k-30 k Da. In some embodiments, the PEG used in the composition has a molecular weight in the range of about 20 k Da.

In some instances, the disclosed hybridization buffer formulations may include the addition of a molecular crowding or volume exclusion agent. Molecular crowding or volume exclusion agents are typically macromolecules (e.g., proteins) which, when added to a solution in high concentrations, may alter the properties of other molecules in solution by reducing the volume of solvent available to the other molecules. In some instances, the percentage by volume of molecular crowding or volume exclusion agent included in the hybridization buffer formulation may range from about 1% to about 50%. In some instances, the percentage by volume of molecular crowding or volume exclusion agent may be at least 1%, at least 5%, at least 10%, at least 15%, at least 20%, at least 25%, at least 30%, at least 35%, at least 40%, at least 45%, or at least 50%. In some instances, the percentage by volume of molecular crowding or volume exclusion agent may be at most 50%, at most 45%, at most 40%, at most 35%, at most 30%, at most 25%, at most 20%, at most 15%, at most 10%, at most 5%, or at most 1%. Any of the lower and upper values described in this paragraph may be combined to form a range included within the present disclosure, for example, the percentage by volume of molecular crowding or volume exclusion agent may range from about 5% to about 35%. Those of skill in the art will recognize that the percentage by volume of molecular crowding or volume exclusion agent may have any value within this range, e.g., about 12.5%.

PH buffer system: The compositions described herein include pH buffer system that maintains the pH of the compositions in a range suitable for hybridization process. The pH buffer system can include one or more buffering agents selected from the group consisting of Tris, HEPES, TAPS, Tricine, Bicine, Bis-Tris, NaOH, KOH, TES, EPPS, MES, and MOPS. The pH buffer system can further include a solvent. A preferred pH buffer system includes MOPS, MES, TAPS, phosphate buffer combined with methanol, acetonitrile, ethanol, isopropanol, butanol, t-butyl alcohol, DMF, DMSO, or any combination therein

The amount of the pH buffer system is effective to maintain the pH of the formulation to be in a range suitable for the hybridization. In some instances, the pH may be at least 3, at least 4, at least 5, at least 6, at least 7, at least 8, at least 9, or at least 10. In some instances, the pH may be at most 10, at most 9, at most 8, at most 7, at most 6, at most 5, at most 4, or at most 3. Any of the lower and upper values described in this paragraph may be combined to form a range included within the present disclosure, for example, the pH of the hybridization buffer may range from about 4 to about 8. Those of skill in the art will recognize that the pH of the hybridization buffer may have any value within this range, e.g., about pH 7.8. In some cases, the pH range is about 3 to about 10. In some instances, the disclosed hybridization buffer formulations may include adjustment of pH over the range of about pH 3 to pH 10, with a preferred buffer range of 5-9.

Additives that impact DNA melting temperatures: The compositions described herein can include one or more additives to allow for better control of the melting temperature of the nucleic acid and enhance the stringency control of the hybridization reaction. Hybridization reactions are usually carried out under the stringent conditions in order to achieve hybridization specificity. In some cases, the additive for controlling melting temperature of nucleic acid is formamide.

The amount of the additive for controlling melting temperature of nucleic acid can vary depending on other agents used in the compositions. The amount of the additive for controlling melting temperature of the nucleic acid is about or more than about 1%, 2%, 3%, 5%, 10%, 15%, 20%, 25%, 30%, 35%, 40%, 50%, 60%, or higher, by volume based on the total volume of the formulation. In some cases, the amount of the additive for controlling melting temperature of the nucleic acid is greater than about 2% by volume based on the total volume of the formulation. In some cases, the amount of the additive for controlling melting temperature of the nucleic acid is greater than 5% by volume based on the total volume of the formulation. In some cases, the amount of the additive for controlling melting temperature of the nucleic acid is lower than about 3%, 5%, 10%, 12.5%, 15%, 20%, 25%, 30%, 35%, 40%, 50%, 60%, 70%, 80%, 90%, or higher, by volume based on the total volume of the formulation. In some embodiments, the amount of the additive for controlling melting temperature of the nucleic acid is in the range of about 1% to 40%, 1% to 35%, 2% to 50%, 2% to 40%, 2% to 35%, 2% to 30%, 2% to 25%, 2% to 20%, 2% to 10%, 5% to 50%, 5% to 40%, 5% to 35%, 5% to 30%, 5% to 25%, 5% to 20%, by volume based on the total volume of the formulation. In some embodiments, the amount of the additive for controlling melting temperature of the nucleic acid is in the range of about 2% to 20% by volume based on the total volume of the formulation. In some cases, the amount of the additive for controlling melting temperature of the nucleic acid is in the range of about 5% to 10% by volume based on the total volume of the formulation.

In some instances, the disclosed hybridization buffer formulations may include the addition of an additive that alters nucleic acid duplex melting temperature. Examples of suitable additives that may be used to alter nucleic acid melting temperature include, but are not limited to, Formamide. In some instances, the percentage by volume of a melting temperature additive included in the hybridization buffer formulation may range from about 1% to about 50%. In some instances, the percentage by volume of a melting temperature additive may be at least 1%, at least 5%, at least 10%, at least 15%, at least 20%, at least 25%, at least 30%, at least 35%, at least 40%, at least 45%, or at least 50%. In some instances, the percentage by volume of a melting temperature additive may be at most 50%, at most 45%, at most 40%, at most 35%, at most 30%, at most 25%, at most 20%, at most 15%, at most 10%, at most 5%, or at most 1%. Any of the lower and upper values described in this paragraph may be combined to form a range included within the present disclosure, for example, the percentage by volume of a melting temperature additive may range from about 10% to about 25%. Those of skill in the art will recognize that the percentage by volume of a melting temperature additive may have any value within this range, e.g., about 22.5%.

Additives that impact DNA hydration: In some instances, the disclosed hybridization buffer formulations may include the addition of an additive that impacts nucleic acid hydration. Examples include, but are not limited to, betaine, urea, glycine betaine, or any combination thereof. In some instances, the percentage by volume of a hydration additive included in the hybridization buffer formulation may range from about 1% to about 50%. In some instances, the percentage by volume of a hydration additive may be at least 1%, at least 5%, at least 10%, at least 15%, at least 20%, at least 25%, at least 30%, at least 35%, at least 40%, at least 45%, or at least 50%. In some instances, the percentage by volume of a hydration additive may be at most 50%, at most 45%, at most 40%, at most 35%, at most 30%, at most 25%, at most 20%, at most 15%, at most 10%, at most 5%, or at most 1%. Any of the lower and upper values described in this paragraph may be combined to form a range included within the present disclosure, for example, the percentage by volume of a hydration additive may range from about 1% to about 30%. Those of skill in the art will recognize that the percentage by volume of a melting temperature additive may have any value within this range, e.g., about 6.5%.

Low non-specific binding surface: Disclosed herein includes a low non-specific binding surface that enable improved nucleic acid hybridization and amplification performance. In general, the disclosed surface may comprise one or more layers of a covalently or non-covalently attached low-binding, chemical modification layers, e.g., silane layers, polymer films, and one or more covalently or non-covalently attached primer sequences that may be used for tethering single-stranded template oligonucleotides to the surface. In some instances, the formulation of the surface, e.g., the chemical composition of one or more layers, the coupling chemistry used to cross-link the one or more layers to the surface and/or to each other, and the total number of layers, may be varied such that non-specific binding of proteins, nucleic acid molecules, and other hybridization and amplification reaction components to the surface is minimized or reduced relative to a comparable monolayer. Often, the formulation of the surface may be varied such that non-specific hybridization on the surface is minimized or reduced relative to a comparable monolayer. The formulation of the surface may be varied such that non-specific amplification on the surface is minimized or reduced relative to a comparable monolayer. The formulation of the surface may be varied such that specific amplification rates and/or yields on the surface are maximized. Amplification levels suitable for detection are achieved in no more than 2, 3, 4, 5, 6, 7, 8, 9, 10, 15, or 30 amplification cycles in some cases disclosed herein.

Examples of materials from which the substrate or support structure may be fabricated include, but are not limited to, glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

The substrate or support structure may be rendered in any of a variety of geometries and dimensions known to those of skill in the art, and may comprise any of a variety of materials known to those of skill in the art. For example, in some instances the substrate or support structure may be locally planar (e.g., comprising a microscope slide or the surface of a microscope slide). Globally, the substrate or support structure may be cylindrical (e.g., comprising a capillary or the interior surface of a capillary), spherical (e.g., comprising the outer surface of a non-porous bead), or irregular (e.g., comprising the outer surface of an irregularly-shaped, non-porous bead or particle). In some instances, the surface of the substrate or support structure used for nucleic acid hybridization and amplification may be a solid, non-porous surface. In some instances, the surface of the substrate or support structure used for nucleic acid hybridization and amplification may be porous, such that the coatings described herein penetrate the porous surface, and nucleic acid hybridization and amplification reactions performed thereon may occur within the pores.

The substrate or support structure that comprises the one or more chemically-modified layers, e.g., layers of a low non-specific binding polymer, may be independent or integrated into another structure or assembly. For example, in some instances, the substrate or support structure may comprise one or more surfaces within an integrated or assembled microfluidic flow cell. The substrate or support structure may comprise one or more surfaces within a microplate format, e.g., the bottom surface of the wells in a microplate. As noted above, in some preferred embodiments, the substrate or support structure comprises the interior surface (such as the lumen surface) of a capillary. In alternate preferred embodiments the substrate or support structure comprises the interior surface (such as the lumen surface) of a capillary etched into a planar chip.

The chemical modification layers may be applied uniformly across the surface of the substrate or support structure. Alternately, the surface of the substrate or support structure may be non-uniformly distributed or patterned, such that the chemical modification layers are confined to one or more discrete regions of the substrate. For example, the substrate surface may be patterned using photolithographic techniques to create an ordered array or random pattern of chemically-modified regions on the surface. Alternately or in combination, the substrate surface may be patterned using, e.g., contact printing and/or ink-jet printing techniques. In some instances, an ordered array or random patter of chemically-modified discrete regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions, or any intermediate number spanned by the range herein.

In order to achieve low nonspecific binding surfaces (also referred to herein as “low binding” or “passivated” surfaces), hydrophilic polymers may be nonspecifically adsorbed or covalently grafted to the substrate or support surface. Typically, passivation is performed utilizing poly(ethylene glycol) (PEG, also known as polyethylene oxide (PEO) or polyoxyethylene), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo (ethylene glycol) methyl ether methacrylate) (POEGMA), polyglutamic acid (PGA), poly-lysine, poly-glucoside, streptavidin, dextran, or other hydrophilic polymers with different molecular weights and end groups that are linked to a surface using, for example, silane chemistry. The end groups distal from the surface can include, but are not limited to, biotin, methoxy ether, carboxylate, amine, NHS ester, maleimide, and bis-silane. In some instances, two or more layers of a hydrophilic polymer, e.g., a linear polymer, branched polymer, or multi-branched polymer, may be deposited on the surface. In some instances, two or more layers may be covalently coupled to each other or internally cross-linked to improve the stability of the resulting surface. In some instances, oligonucleotide primers with different base sequences and base modifications (or other biomolecules, e.g., enzymes or antibodies) may be tethered to the resulting surface layer at various surface densities. In some instances, for example, both surface functional group density and oligonucleotide concentration may be varied to target a certain primer density range. Additionally, primer density can be controlled by diluting oligonucleotide with other molecules that carry the same functional group. For example, amine-labeled oligonucleotide can be diluted with amine-labeled polyethylene glycol in a reaction with an NHS-ester coated surface to reduce the final primer density. Primers with different lengths of linker between the hybridization region and the surface attachment functional group can also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

As a result of the surface passivation techniques disclosed herein, proteins, nucleic acids, and other biomolecules do not “stick” to the substrates, that is, they exhibit low nonspecific binding (NSB). Examples are shown below using standard monolayer surface preparations with varying glass preparation conditions. Hydrophilic surface that have been passivated to achieve ultra-low NSB for proteins and nucleic acids require novel reaction conditions to improve primer deposition reaction efficiencies, hybridization performance, and induce effective amplification. All of these processes require oligonucleotide attachment and subsequent protein binding and delivery to a low binding surface. As described below, the combination of a new primer surface conjugation formulation (Cy3 oligonucleotide graft titration) and resulting ultra-low non-specific background (NSB functional tests performed using red and green fluorescent dyes) yielded results that demonstrate the viability of the disclosed approaches. Some surfaces disclosed herein exhibit a ratio of specific (e.g., hybridization to a tethered primer or probe) to nonspecific binding (e.g., Bᵢₙₜₑᵣ) of a fluorophore such as Cy3 of at least 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 50:1, 75:1, 100:1, or greater than 100:1, or any intermediate value spanned by the range herein. Some surfaces disclosed herein exhibit a ratio of specific to nonspecific fluorescence signal (e.g., for specifically-hybridized to nonspecifically bound labeled oligonucleotides, or for specifically-amplified to nonspecifically-bound (Bᵢₙₜₑᵣ) or non-specifically amplified (Bᵢₙₜᵣₐ) labeled oligonucleotides or a combination thereof (Bᵢₙₜₑᵣ+Bᵢₙₜᵣₐ)) for a fluorophore such as Cy3 of at least 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 50:1, 75:1, 100:1, or greater than 100:1, or any intermediate value spanned by the range herein.

In order to scale primer surface density and add additional dimensionality to hydrophilic or amphoteric surfaces, substrates comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the surface significantly. Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some instances, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some instances, high primer density materials may be constructed in solution and subsequently layered onto the surface in multiple steps.

The attachment chemistry used to graft a first chemically-modified layer to a support surface will generally be dependent on both the material from which the support is fabricated and the chemical nature of the layer. In some instances, the first layer may be covalently attached to the support surface. In some instances, the first layer may be non-covalently attached, e.g., adsorbed to the surface through non-covalent interactions such as electrostatic interactions, hydrogen bonding, or van der Waals interactions between the surface and the molecular components of the first layer. In either case, the substrate surface may be treated prior to attachment or deposition of the first layer. Any of a variety of surface preparation techniques known to those of skill in the art may be used to clean or treat the support surface. For example, glass or silicon surfaces may be acid-washed using a Piranha solution (a mixture of sulfuric acid (H₂SO₄) and hydrogen peroxide (H₂O₂)) and/or cleaned using an oxygen plasma treatment method.

Silane chemistries constitute one non-limiting approach for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding support surfaces include, but are not limited to, (3-Aminopropyl) trimethoxysilane (APTMS), (3-Aminopropyl) triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the support surface, where the choice of components used may be varied to alter one or more properties of the support surface, e.g., the surface density of functional groups and/or tethered oligonucleotide primers, the hydrophilicity/hydrophobicity of the support surface, or the three three-dimensional nature (i.e., “thickness”) of the support surface. Examples of preferred polymers that may be used to create one or more layers of low non-specific binding material in any of the disclosed support surfaces include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the support surface and/or to cross-link the layers to each other include, but are not limited to, biotin-streptavidin interactions (or variations thereof), his tag-Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

One or more layers of a multi-layered surface may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branced PHEMA), branched poly(oligo (ethylene glycol) methyl ether methacrylate) (branched POEGMA), branched polyglutamic acid (branched PGA), branched poly-lysine, branched poly-glucoside, and dextran.

In some instances, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branches. Molecules often exhibit a ‘power of 2’ number of branches, such as 2, 4, 8, 16, 32, 64, or 128 branches.

Exemplary PEG multilayers include PEG (8,16,8) on PEGamine-APTES, exposed to two layers of 7 uM primer pre-loading, exhibited a concentration of 2,000,000 to 10,000,000 on the surface. Similar concentrations were observed for 3-layer multi-arm PEG (8,16,8) and (8,64,8) on PEGamine-APTES exposed to 8 uM primer, and 3-layer multi-arm PEG (8,8,8) using star-shape PEG-amine to replace dumbbell-shaped 16mer and 64mer. PEG multilayers having comparable first, second and third PEG level are also contemplated.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some instances, e.g., wherein at least one layer of a multi-layered surface comprises a branched polymer, the number of covalent bonds between a branched polymer molecule of the layer being deposited and molecules of the previous layer may range from about one covalent linkages per molecule and about 32 covalent linkages per molecule. In some instances, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 or more than 32 covalent linkages per molecule.

Any reactive functional groups that remain following the coupling of a material layer to the support surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface of the disclosed low binding supports may range from 1 to about 10. In some instances, the number of layers is at least 1, at least 2, at least 3, at least 4, at least 5, at least 6, at least 7, at least 8, at least 9, or at least 10. In some instances, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some instances the number of layers may range from about 2 to about 4. In some instances, all of the layers may comprise the same material. In some instances, each layer may comprise a different material. In some instances, the plurality of layers may comprise a plurality of materials. In some instances at least one layer may comprise a branched polymer. In some instance, all of the layers may comprise a branched polymer.

One or more layers of low non-specific binding material may in some cases be deposited on and/or conjugated to the substrate surface using a polar protic solvent, a polar aprotic solvent, a nonpolar solvent, or any combination thereof. In some instances the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino) propanesulfonic acid (MOPS), etc.), or any combination thereof. In some instances, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, or any percentage spanned or adjacent to the range herein, with the balance made up of water or an aqueous buffer solution. In some instances, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, or any percentage spanned or adjacent to the range herein, with the balance made up of an organic solvent. The pH of the solvent mixture used may be less than 5, 5, 5, 5, 6, 6, 6.5, 7, 7.5, 8, 8.5, 9, 9.5, 10, or greater than 10, or any value spanned or adjacent to the range described herein.

In some instances, one or more layers of low non-specific binding material may be deposited on and/or conjugated to the substrate surface using a mixture of organic solvents, wherein the dielectric constant of at least once component is less than 40 and constitutes at least 50% of the total mixture by volume. In some instances, the dielectric constant of the at least one component may be less than 10, less than 20, less than 30, less than 40. In some instances, the at least one component constitutes at least 20%, at least 30%, at least 40%, at least 50%, at least 50%, at least 60%, at least 70%, or at least 80% of the total mixture by volume.

As noted, the low non-specific binding supports of the present disclosure exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, in some instances, exposure of the surface to fluorescent dyes (e.g., cyanine dye 3 (Cy3), cyanine dye 5 (Cy5), etc.), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding on supports comprising different surface formulations. In some instances, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations-provided that care has been taken to ensure that the fluorescence imaging is performed under conditions where fluorescence signal is linearly related (or related in a predictable manner) to the number of fluorophores on the support surface (e.g., under conditions where signal saturation and/or self-quenching of the fluorophore is not an issue) and suitable calibration standards are used. In some instances, other techniques known to those of skill in the art, for example, radioisotope labeling and counting methods may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

As noted, in some instances, the degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some instances, the label may comprise a fluorescent label. In some instances, the label may comprise a radioisotope. In some instances, the label may comprise any other detectable label known to one of skill in the art. In some instances, the degree of non-specific binding exhibited by a given support surface formulation may thus be assessed in terms of the number of non-specifically bound protein molecules (or other molecules) per unit area. In some instances, the low-binding supports of the present disclosure may exhibit non-specific protein binding (or non-specific binding of other specified molecules, e.g., Cy3 dye) of less than 0.001 molecule per μm², less than 0.01 molecule per μm², less than 0.1 molecule per μm², less than 0.25 molecule per μm², less than 0.5 molecule per μm², less than Imolecule per μm², less than 10 molecules per μm², less than 100 molecules per μm², or less than 1,000 molecules per μm². Those of skill in the art will realize that a given support surface of the present disclosure may exhibit non-specific binding falling anywhere within this range, for example, of less than 86 molecules per μm². For example, some modified surfaces disclosed herein exhibit nonspecific protein binding of less than 0.5 molecule/μm² following contact with a 1 uM solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm². In independent nonspecific binding assays, 1 uM labeled Cy3 SA (ThermoFisher), 1 uM Cy5 SA dye (ThermoFisher), 10 uM Aminoallyl-dUTP-ATTO-647N (Jena Biosciences), 10 uM Aminoallyl-dUTP-ATTO-Rholl (Jena Biosciences), 10 uM Aminoallyl-dUTP-ATTO-Rholl (Jena Biosciences), 10 uM 7-Propargylamino-7-deaza-dGTP-Cy5 (Jena Biosciences, and 10 uM 7-Propargylamino-7-deaza-dGTP-Cy3 (Jena Biosciences) were incubated on the low binding substrates at 37° C. for 15 minutes in a 384 well plate format. Each well was rinsed 2-3× with 50 ul deionized RNase/DNase Free water and 2-3× with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 μm. For higher resolution imaging, images were collected on an Olympus IX83 microscope (Olympus Corp., Center Valley, PA) with a total internal reflectance fluorescence (TIRF) objective (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, New York), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per μm².

In some instances, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some instances, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some instances, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some instances, a static contact angle may be determined. In some instances, an advancing or receding contact angle may be determined. In some instances, the water contact angle for the hydrophilic, low-binding support surfaced disclosed herein may range from about 0 degrees to about 30 degrees. In some instances, the water contact angle for the hydrophilic, low-binding support surfaced disclosed herein may no more than 50 degrees, 45 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

In some instances, the hydrophilic surfaces disclosed herein facilitate reduced wash times for bioassays, often due to reduced nonspecific binding of biomolecules to the low-binding surfaces. In some instances, adequate wash steps may be performed in less than 60, 50, 40, 30, 20, 15, 10, or less than 10 seconds. For example, in some instances adequate wash steps may be performed in less than 30 seconds.

Some low-binding surfaces of the present disclosure exhibit significant improvement in stability or durability to prolonged exposure to solvents and elevated temperatures, or to repeated cycles of solvent exposure or changes in temperature. For example, in some instances, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some instances, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some instances, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some instances, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100 fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100 fold greater than a signal of an adjacent amplified nucleic acid population region of the surface.

Fluorescence excitation energies vary among particular fluorophores and protocols, and may range in excitation wavelength from less than 400 nm to over 800 nm, consistent with fluorophore selection or other parameters of use of a surface disclosed herein.

Accordingly, low background surfaces as disclosed herein exhibit low background fluorescence signals or high contrast to noise (CNR) ratios relative to known surfaces in the art. For example, in some instances, the background fluorescence of the surface at a location that is spatially distinct or removed from a labeled feature on the surface (e.g., a labeled spot, cluster, discrete region, sub-section, or subset of the surface) comprising a hybridized cluster of nucleic acid molecules, or a clonally-amplified cluster of nucleic acid molecules produced by 20 cycles of nucleic acid amplification via thermocycling, may be no more than 20×, 10×, 5×, 2×, 1×, 0.5×, 0.1×, or less than 0.1× greater than the background fluorescence measured at that same location prior to performing said hybridization or said 20 cycles of nucleic acid amplification.

In some instances, fluorescence images of the disclosed low background surfaces when used in nucleic acid hybridization or amplification applications to create clusters of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

The surface that comprises the one or more chemically-modified layers, e.g., layers of a low non-specific binding polymer, may be independent or integrated into another structure or assembly. The chemical modification layers may be applied uniformly across the surface. Alternately, the surface may be patterned, such that the chemical modification layers are confined to one or more discrete regions of the substrate. For example, the surface may be patterned using photolithographic techniques to create an ordered array or random pattern of chemically-modified regions on the surface. Alternately or in combination, the substrate surface may be patterned using, e.g., contact printing and/or ink-jet printing techniques. In some instances, an ordered array or random patter of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In order to achieve low nonspecific binding surfaces (also referred to herein as “low binding” or “passivated” surfaces), hydrophilic polymers may be nonspecifically adsorbed or covalently grafted to the surface. Typically, passivation is performed utilizing poly(ethylene glycol) (PEG, also known as polyethylene oxide (PEO) or polyoxyethylene) or other hydrophilic polymers with different molecular weights and end groups that are linked to a surface using, for example, silane chemistry. The end groups distal from the surface can include, but are not limited to, biotin, methoxy ether, carboxylate, amine, NHS ester, maleimide, and bis-silane. In some instances, two or more layers of a hydrophilic polymer, e.g., a linear polymer, branched polymer, or multi-branched polymer, may be deposited on the surface. In some instances, two or more layers may be covalently coupled to each other or internally cross-linked to improve the stability of the resulting surface. In some instances, oligonucleotide primers with different base sequences and base modifications (or other biomolecules, e.g., enzymes or antibodies) may be tethered to the resulting surface layer at various surface densities. In some instances, for example, both surface functional group density and oligonucleotide concentration may be varied to target a certain primer density range. Additionally, primer density can be controlled by diluting oligonucleotide with other molecules that carry the same functional group. For example, amine-labeled oligonucleotide can be diluted with amine-labeled polyethylene glycol in a reaction with an NHS-ester coated surface to reduce the final primer density. Primers with different lengths of linker between the hybridization region and the surface attachment functional group can also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

In order to scale primer surface density and add additional dimensionality to hydrophilic or amphoteric surfaces, surfaces comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the surface significantly. Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some instances, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some instances, high primer density materials may be constructed in solution and subsequently layered onto the surface in multiple steps.

The attachment chemistry used to graft a first chemically-modified layer to a surface will generally be dependent on both the material from which the surface is fabricated and the chemical nature of the layer. In some instances, the first layer may be covalently attached to the surface. In some instances, the first layer may be non-covalently attached, e.g., adsorbed to the surface through non-covalent interactions such as electrostatic interactions, hydrogen bonding, or van der Waals interactions between the surface and the molecular components of the first layer. In either case, the substrate surface may be treated prior to attachment or deposition of the first layer. Any of a variety of surface preparation techniques known to those of skill in the art may be used to clean or treat the surface. For example, glass or silicon surfaces may be acid-washed using a Piranha solution (a mixture of sulfuric acid (H₂SO₄) and hydrogen peroxide (H₂O₂)), base treatment in KOH and NaOH, and/or cleaned using an oxygen plasma treatment method.

Silane chemistries constitute one non-limiting approach for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding surfaces include, but are not limited to, (3-Aminopropyl) trimethoxysilane (APTMS), (3-Aminopropyl) triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the surface, where the choice of components used may be varied to alter one or more properties of the surface, e.g., the surface density of functional groups and/or tethered oligonucleotide primers, the hydrophilicity/hydrophobicity of the surface, or the three three-dimensional nature (i.e., “thickness”) of the surface. Examples of preferred polymers that may be used to create one or more layers of low non-specific binding material in any of the disclosed surfaces include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag-Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

One or more layers of a multi-layered surface may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branced PHEMA), branched poly(oligo (ethylene glycol) methyl ether methacrylate) (branched POEGMA), branched polyglutamic acid (branched PGA), branched poly-lysine, branched poly-glucoside, and dextran.

In some instances, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branches.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some instances, e.g., wherein at least one layer of a multi-layered surface comprises a branched polymer, the number of covalent bonds between a branched polymer molecule of the layer being deposited and molecules of the previous layer may range from about one covalent linkages per molecule and about 32 covalent linkages per molecule. In some instances, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some instances, the number of layers is at least 1, at least 2, at least 3, at least 4, at least 5, at least 6, at least 7, at least 8, at least 9, or at least 10. In some instances, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some instances the number of layers may range from about 2 to about 4. In some instances, all of the layers may comprise the same material. In some instances, each layer may comprise a different material. In some instances, the plurality of layers may comprise a plurality of materials. In some instances at least one layer may comprise a branched polymer. In some instance, all of the layers may comprise a branched polymer.

One or more layers of low non-specific binding material may in some cases be deposited on and/or conjugated to the substrate surface using a polar protic solvent, an organic solvent, a nonpolar solvent, or any combination thereof. In some instances the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino) propanesulfonic acid (MOPS), etc.), or any combination thereof. In some instances, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some instances, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than 9 mk.

As noted, the low non-specific binding surface exhibit reduced non-specific binding of nucleic acids, and other components of the hybridization and/or amplification formulation used for solid-phase nucleic acid amplification. The degree of non-specific binding exhibited by a given surface may be assessed either qualitatively or quantitatively. For example, in some instances, exposure of the surface to fluorescent dyes (e.g., Cy3, Cy5, etc.), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding surface comprising different surface formulations. In some instances, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on surfaces comprising different surface formulations-provided that care has been taken to ensure that the fluorescence imaging is performed under conditions where fluorescence signal is linearly related (or related in a predictable manner) to the number of fluorophores on the surface (e.g., under conditions where signal saturation and/or self-quenching of the fluorophore is not an issue) and suitable calibration standards are used. In some instances, other techniques known to those of skill in the art, for example, radioisotope labeling and counting methods may be used for quantitative assessment of the degree to which non-specific binding is exhibited by the different surface formulations of the present disclosure.

As noted, in some instances, the degree of non-specific binding exhibited by the disclosed low-binding surfaces may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some instances, the label may comprise a fluorescent label. In some instances, the label may comprise a radioisotope. In some instances, the label may comprise any other detectable label known to one of skill in the art. In some instances, the degree of non-specific binding exhibited by a given surface formulation may thus be assessed in terms of the number of non-specifically bound protein molecules (or other molecules) per unit area. In some instances, the low-binding surfaces of the present disclosure may exhibit non-specific protein binding (or non-specific binding of other specified molecules, e.g., Cy3 dye) of less than 0.001 molecule per μm², less than 0.01 molecule per μm², less than 0.1 molecule per μm², less than 0.25 molecule per μm², less than 0.5 molecule per μm², less than 1 molecule per μm², less than 10 molecules per μm², less than 100 molecules per μm², or less than 1,000 molecules per μm². Those of skill in the art will realize that a given surface of the present disclosure may exhibit non-specific binding falling anywhere within this range, for example, of less than 86 molecules per μm². For example, some modified surfaces disclosed herein exhibit nonspecific protein binding of less than 0.5 molecule/μm² following contact with a 1 μM solution of bovine serum albumin (BSA) in phosphate buffered saline (PBS) buffer for 30 minutes, followed by a 10 minute PBS rinse. Some modified surfaces disclosed herein exhibit nonspecific binding of Cy3 dye molecules of less than 0.25 molecules per μm².

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some instances, the degree of hydrophilicity (or “wettability” with aqueous solutions) of the disclosed surfaces may be assessed, for example, through the measurement of water contact angles in which a small droplet of water is placed on the surface and its angle of contact with the surface is measured using, e.g., an optical tensiometer. In some instances, a static contact angle may be determined. In some instances, an advancing or receding contact angle may be determined. In some instances, the water contact angle for the hydrophilic, low-binding surfaces disclosed herein may range from about 0 degrees to about 30 degrees. In some instances, the water contact angle for the hydrophilic, low-binding surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding surface of the present disclosure may exhibit a water contact angle having a value of anywhere within this range.

In some instances, the low-binding surfaces of the present disclosure may exhibit significant improvement in stability or durability to prolonged exposure to solvents and elevated temperatures, or to repeated cycles of solvent exposure or changes in temperature. For example, in some instances, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some instances, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some instances, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some instances, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100 fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100 fold greater than a signal of an adjacent amplified nucleic acid population region of the surface.

Accordingly, low background surfaces as disclosed herein exhibit low background fluorescence signals or high contrast to noise (CNR) ratios relative to known surfaces in the art.

Oligonucleotide primers and adapter sequences: In general, at least one layer of the one or more layers of low non-specific binding material may comprise functional groups for covalently or non-covalently attaching oligonucleotide adapter or primer sequences, or the at least one layer may already comprise covalently or non-covalently attached oligonucleotide adapter or primer sequences at the time that it is deposited on the support surface. In some instances, the oligonucleotides tethered to the polymer molecules of at least one third layer may be distributed at a plurality of depths throughout the layer.

One or more types of oligonucleotide primer may be attached or tethered to the support surface. In some instances, the one or more types of oligonucleotide adapters or primers may comprise spacer sequences, adapter sequences for hybridization to adapter-ligated template library nucleic acid sequences, forward amplification primers, reverse amplification primers, sequencing primers, and/or molecular barcoding sequences, or any combination thereof. In some instances, 1 primer or adapter sequence may be tethered to at least one layer of the surface. In some instances, at least 2, 3, 4, 5, 6, 7, 8, 9, 10, or more than 10 different primer or adapter sequences may be tethered to at least one layer of the surface.

In some instances, the tethered oligonucleotide adapter and/or primer sequences may range in length from about 10 nucleotides to about 100 nucleotides. In some instances, the tethered oligonucleotide adapter and/or primer sequences may be at least 10, at least 20, at least 30, at least 40, at least 50, at least 60, at least 70, at least 80, at least 90, or at least 100 nucleotides in length. In some instances, the tethered oligonucleotide adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some instances the length of the tethered oligonucleotide adapter and/or primer sequences may range from about 20 nucleotides to about 80 nucleotides. Those of skill in the art will recognize that the length of the tethered oligonucleotide adapter and/or primer sequences may have any value within this range, e.g., about 24 nucleotides.

In some instances, the tethered primer sequences may comprise modifications designed to facilitate the specificity and efficiency of nucleic acid amplification as performed on the low-binding supports. For example, in some instances the primer may comprise polymerase stop points such that the stretch of primer sequence between the surface conjugation point and the modification site is always in single-stranded form and functions as a loading site for 5′ to 3′ helicases in some helicase-dependent isothermal amplification methods. Other examples of primer modifications that may be used to create polymerase stop points include, but are not limited to, an insertion of a PEG chain into the backbone of the primer between two nucleotides towards the 5′ end, insertion of an abasic nucleotide (i.e., a nucleotide that has neither a purine nor a pyrimidine base), or a lesion site which can be bypassed by the helicase.

As will be discussed further in the examples below, it may be desirable to vary the surface density of tethered primers on the support surface and/or the spacing of the tethered primers away from the support surface (e.g., by varying the length of a linker molecule used to tether the primers to the surface) in order to “tune” the support for optimal performance when using a given amplification method. As noted below, adjusting the surface density of tethered primers may impact the level of specific and/or non-specific amplification observed on the support in a manner that varies according to the amplification method selected. In some instances, the surface density of tethered oligonucleotide primers may be varied by adjusting the ratio of molecular components used to create the support surface. For example, in the case that an oligonucleotide primer-PEG conjugate is used to create the final layer of a low-binding support, the ratio of the oligonucleotide primer-PEG conjugate to a non-conjugated PEG molecule may be varied. The resulting surface density of tethered primer molecules may then be estimated or measured using any of a variety of techniques known to those of skill in the art. Examples include, but are not limited to, the use of radioisotope labeling and counting methods, covalent coupling of a cleavable molecule that comprises an optically-detectable tag (e.g., a fluorescent tag) that may be cleaved from a support surface of defined area, collected in a fixed volume of an appropriate solvent, and then quantified by comparison of fluorescence signals to that for a calibration solution of known optical tag concentration, or using fluorescence imaging techniques provided that care has been taken with the labeling reaction conditions and image acquisition settings to ensure that the fluorescence signals are linearly related to the number of fluorophores on the surface (e.g., that there is no significant self-quenching of the fluorophores on the surface).

In some instances, the resultant surface density of oligonucleotide primers on the low binding support surfaces of the present disclosure may range from about 1,000 primer molecules per μm² to about 1,000,000 primer molecules per μm². In some instances, the surface density of oligonucleotide primers may be at least 1,000, at least 10,000, at least 100,000, or at least 1,000,000 molecules per μm². In some instances, the surface density of oligonucleotide primers may be at most 1,000,000, at most 100,000, at most 10,000, or at most 1,000 molecules per μm². Any of the lower and upper values described in this paragraph may be combined to form a range included within the present disclosure, for example, in some instances the surface density of primers may range from about 10,000 molecules per μm² to about 100,000 molecules per μm². Those of skill in the art will recognize that the surface density of primer molecules may have any value within this range, e.g., about 455,000 molecules per μm². In some instances, the surface density of template library nucleic acid sequences initially hybridized to adapter or primer sequences on the support surface may be less than or equal to that indicated for the surface density of tethered oligonucleotide primers. In some instances, the surface density of clonally-amplified template library nucleic acid sequences hybridized to adapter or primer sequences on the support surface may span the same range as that indicated for the surface density of tethered oligonucleotide primers.

Local densities as listed above do not preclude variation in density across a surface, such that a surface may comprise a region having an oligo density of, for example, 500,000/μm², while also comprising at least a second region having a substantially different local density.

Improvements in hybridization rate: In some instances, the use of the buffer formulations disclosed herein (optionally, used in combination with low non-specific binding surface) yield relative hybridization rates that range from about 2× to about 20× faster than that for a conventional hybridization protocol. In some instances, the relative hybridization rate may be at least 2×, at least 3×, at least 4×, at least 5×, at least 6×, at least 7×, at least 8×, at least 9×, at least 10×, at least 12×, at least 14×, at least 16×, at least 18×, or at least 20× that for a conventional hybridization protocol.

The method and composition described herein can help shorten the time required for completing the hybridization step. In some embodiments, the hybridization time can be in the range of about 1 s to 2 h, about 5 s to 1.5 h, about 15 s to 1 h, or about 15 s to 0.5 h. In some embodiments, the hybridization time can be in the range of about 15 s to 1 h. In some embodiments, the hybridization time can be shorter than 15 s, 30 s, 1 min, 1.5 min, 2 min, 2.5 min, 3 min, 4 min, 5 min, 6 min, 7 min, 8 min, 9 min, 10 min, 15 min, 20 min, 25 min, 30 min, 40 min, 50 min, 60 min, 70 min, 80 min, 90 min, 100 min, 110 min, or 120 min. In some embodiments, the hybridization time can be longer than 1 s, 5 s, 10 s, 15 s, 30 s, 1 min, 1.5 min, 2 min, 2.5 min, 3 min, 4 min, or 5 min.

The annealing methods described herein can significantly shorten the annealing time. In some embodiments, at least 90% of the target nucleic acid anneals to the surface bound nucleic acid in less than 15 s, 30 s, 1 min, 1.5 min, 2 min, 2.5 min, 3 min, 4 min, 5 min, 6 min, 7 min, 8 min, 9 min, 10 min, 15 min, 20 min, 25 min, 30 min, 40 min, 50 min, 60 min, 70 min, 80 min, 90 min, 100 min, 110 min, or 120 min. In some embodiments, at least 80% of the target nucleic acid anneals to the surface bound nucleic acid in less than 15 s, 30 s, 1 min, 1.5 min, 2 min, 2.5 min, 3 min, 4 min, 5 min, 6 min, 7 min, 8 min, 9 min, 10 min, 15 min, 20 min, 25 min, 30 min, 40 min, 50 min, 60 min, 70 min, 80 min, 90 min, 100 min, 110 min, or 120 min. In some embodiments, at least 90% of the target nucleic acid anneals to the surface bound nucleic acid in greater than Is, 5 s, 10 s, 15 s, 30 s, 1 min, 1.5 min, 2 min, 2.5 min, 3 min, 4 min, or 5 min. In some embodiments, at least 90% of the target nucleic acid anneals to the surface bound nucleic acid in the range of about 10 s to about 1 hour, about 30 s to about 50 min, about 1 min to about 50 min, or about 1 min to about 30 min.

Improvements in hybridization efficiency: As used herein, hybridization efficiency (or yield) is a measure of the percentage of total available tethered adapter sequences on a solid surface, primer sequences, or oligonucleotide sequences in general that are hybridized to complementary sequences. In some instances, the use of optimized buffer formulations disclosed herein (optionally, used in combination with low non-specific binding surface) yield improved hybridization efficiency compared to that for a conventional hybridization protocol. In some instances, the hybridization efficiency that may be achieved is better than 80%, 85%, 90%, 95%, 98%, or 99% in any of the hybridization reaction times specified above.

The method and composition described herein can be used in an isothermal annealing conditions. In some embodiments, the methods described herein can eliminate the cooling step required for most hybridization step. In some embodiments, the annealing methods described herein can be performed at a temperature in the range of about 10° C. to 95° C., about 20° C. to 80° C., about 30° C. to 70° C. In some embodiments, the temperature can be lower than about 40° C., 50° C., 60° C., 70° C., 80° C., or 90° C.

Improvements in hybridization specificity: As used herein, hybridization specificity is a measure of the ability of tethered adapter sequences, primer sequences, or oligonucleotide sequences in general to correctly hybridize only to completely complementary sequences. In some instances, the use of the optimized buffer formulations disclosed herein (optionally, used in combination with low non-specific binding surface) yield improved hybridization specificity compared to that for a conventional hybridization protocol. In some instances, the hybridization specificity that may be achieved is better than 1 base mismatch in 10 hybridization events, 1 base mismatch in 100 hybridization events, 1 base mismatch in 1,000 hybridization events, or 1 base mismatch in 10,000 hybridization events.

### EXAMPLES

These examples are provided for illustrative purposes only and not to limit the scope of the claims provided herein.

### Example 1—DNA Hybridization on Low Non-Specific Binding Surface

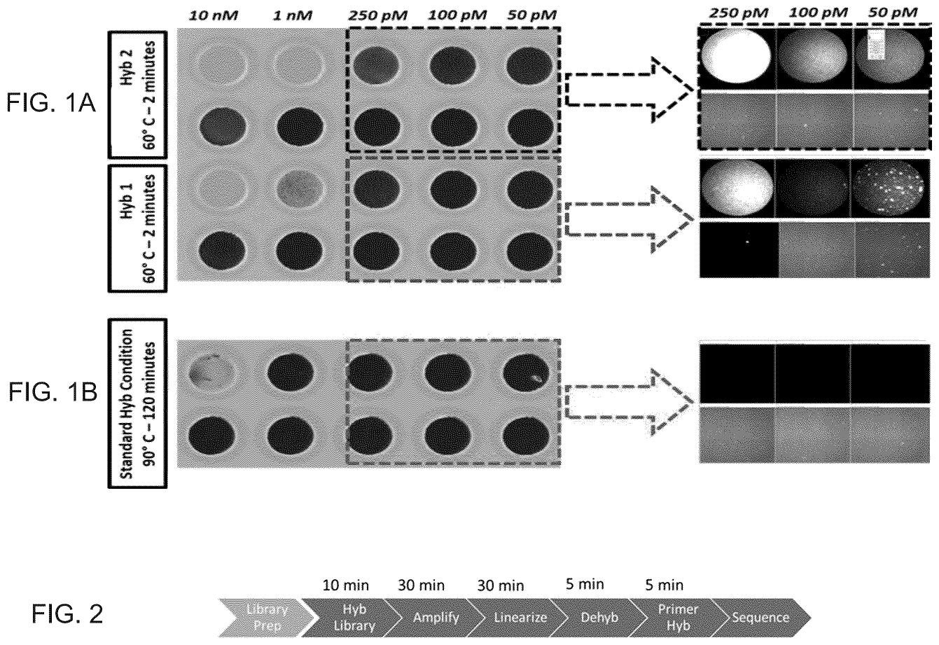

FIGS. 1A-B provide examples of the improved hybridization achieved on low binding surface using the disclosed hybridization method (FIG. 1A) with reduced amounts of input DNA and shortened hybridization times, as compared to the results achieved using a conventional hybridization protocol (FIG. 1B).

Traditional or standard conditions were tested with hybridization reporter probe (complementary oligonucleotide sequences labeled with a Cy™3 fluorophore at the 5′ end) in 2×-5× saline-sodium citrate (SSC) buffer (std) at concentrations reported at 90 degree with a slow cool process (2 hours) to reach 37 degrees. The surfaces used for both testing conditions were ultra-low non-specific binding surfaces having a level of non-specific Cy3 dye absorption of less than about 0.25 molecules/μm². Wells were washed with 50 mM Tris pH 8.0; 50 mM NaCl. Images were obtained acquired using an inverted microscope (Olympus IX83) equipped with 100×TIRF objective, NA=1.4 (Olympus), dichroic mirror optimized for 532 nm light (Semrock, Di03-R532-t1-25×36), a bandpass filter optimized for Cy3 emission, (Semrock, FF01-562/40-25), and a camera (sCMOS, Andor Zyla) under non-signal saturating conditions for 1 s, (Laser Quantum, Gem 532, <1 W/cm² at the sample) while sample is immersed a buffer (25 mM ACES, pH 7.4 buffer). From Table 1, conditions C10 and D18 in Table 1 were chosen to test the viability of these conditions to improve existing standard surface hybridization protocols on a low binding substrate. Condition 1 and 2 were chosen from Table 1. The oligonucleotide probe was added at concentrations specified and hybridization performed for 2 min at 50 degrees C. Images were collected as described above and results shown (FIGS. 1A and 1B).

FIG. 2 illustrates a workflow for nucleic acid sequencing using the disclosed hybridization methods on low binding surfaces, and non-limiting examples of the processing times that may be achieved.

### Example 2

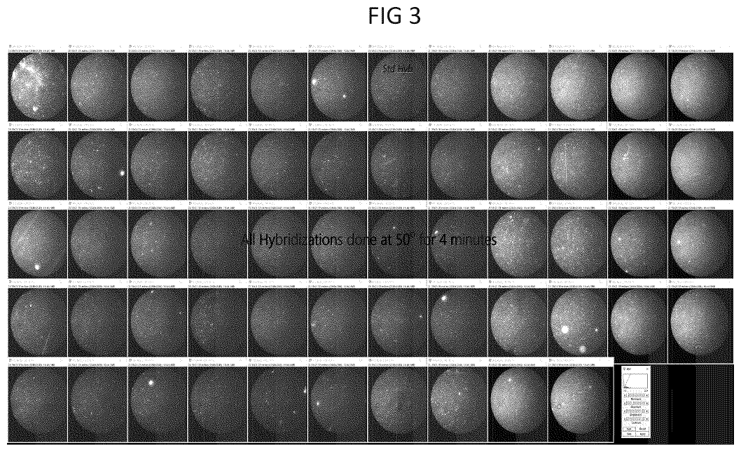

Glass substrates (175 um 22×60 mm², Corning Glass) were cleaned with KOH and ethanol. Low binding glass surfaces were prepared by incubating Silane-PEG5K-NHS (Nanocs) in ethanol at 65 degrees for 30 minutes. Oligonucleotides with 5′ modified NH2 were grafted to these surfaces in a mixture of 1 uM oligos in methanol/phosphate buffer for 20 minutes. Monotemplate oligos fragments (approximately 100 bp) were circularized using splint ligation protocol that contained complementary fragments to surface grafted primers. Following circularization of library, circular library fragments were added at 100 pM in various hybridization test mixtures. Individual buffer/library hybridization mixtures were added to 384 well plate with the functionalized surface affixed at 50 degrees for 4 minutes. Intercalating DNA stain was added to visualize the effectiveness of each of the individual buffer compositions for fast and specific hybridization of the circularized libraries. The 384 well plate was imaged using a fluorescence microscope and 488 nm excitation with a 60× water immersion objective (1.2 NA, Olympus) (FIG. 3). A number of compositions were tested for the hybridization of target nucleic acid with surface bound nucleic acid. Table 1 below lists the compositions that were tested.

TABLE 1
Buffer compositions tested for hybridizing target nucleic acid with surface bound nucleic acid

| Graft | | | | | | | | | | | | | |
|---|---|---|---|---|---|---|---|---|---|---|---|---|---|
| concentration | | 1 uM | | | | | | | | 5.1 uM | | 46 uM | |
| | 9 | 10 | 11 | 12 | 13 | 14 | 15 | 16 | 17 | 18 | 19 | 20 | 21 |
| B | Cracked | 75% | 75% | 2 × SSC | 25% | Std | 30% | Std | 50% | Std | Std | Std | Std |
| | | ACN + | ACN + | | ACN + | buf. + | PEG | | ACN + | | | | |
| | | MES | Phos | | 2 × SSC + | 5% | | | 50% | | | | |
| | | | | | 10% | PEG + | | | Std | | | | |
| | | | | | PEG | 30% | | | buf. | | | | |
| | | | | | | Form. | | | | | | | |
| C | 1 uM | 50% | 50% | 4 × SSC | 25% | Std | 20% | Std + 2 | Std + 2 | Tris + | Tris + | Std | Std |
| | 31-NH2- | ACN + | ACN + | | ACN + | buf. + | PEG + | | | 1 × SSC | 1 × SSC | buff + | buff + |
| | Cy3 | MES | Tris | | MES + | 10% | 2 × SSC | | | | | 5% | 5% |
| | | | | | 20% | PEG + | | | | | | PEG + | PEG + |
| | | | | | PEG + | 5% | | | | | | 30% | 30% |
| | | | | | 10% | Form. | | | | | | Form. | Form. |
| | | | | | Form. | | | | | | | | |
| D | 1 uM | 25% | 25% | 10 × SSC | 50% | Std | 10% | Std + 4 | Std + 4 | 25% | 25% | Std | Std |
| | 31-NH2- | ACN + | ACN + | | EtOH + | buf. + | PEG + | | | ACN + | ACN + | buff + | buff + |
| | Cy3 | MES + | Tris + | | 2 × SSC | 10% | 2 × SSC + | | | MES + | MES + | 10% | 10% |
| | | 2 × SSC | 2 × SSC | | | PEG + | 5% | | | 20% | 20% | PEG + | PEG + |
| | | | | | | 10% | Form. | | | PEG + | PEG + | 5% | 5% |
| | | | | | | Form. | | | | 10% | 10% | Form. | Form. |
| | | | | | | | | | | Form. | Form. | | |
| E | 1 uM | MES + | Tris + | 20 × SSC | 50% | Std | 5% | Std + 6 | Std + 6 | Std | Std | 10% | 10% |
| | 31-NH2- | 1 × SSC | 1 × SSC | | EtOH + | buf. + | Form. + | | | buf. + | buf. + | PEG + | PEG + |
| | Cy3 | | | | 2 × SSC + | 20% | 2 × SSC | | | 20% | 20% | 2 × SSC + | 2 × SSC + |
| | | | | | 10% | PEG + | | | | PEG + | PEG + | 5% | 5% |
| | | | | | PEG | 10% | | | | 10% | 10% | Form. | Form. |
| | | | | | | Form. | | | | Form. | Form. | | |
| F | 10 nM | 10 nM | 10 nM | 10 × SSC + | Std | Std | 10% | Std + 8 | Std + 8 | Std | Std | 10% | 10% |
| | 31-NH2- | 31-NH2- | 31-NH2- | 10% | | buf. + | Form. + | | | buf. + | buf. + | Form. + | Form. + |
| | Cy3 | Cy3 | Cy3 | Form. | | 10% | 2 × SSC | | | 10% | 10% | 2 × SSC | 2 × SSC |
| | | | | | | Form. | | | | Form. | Form. | | |

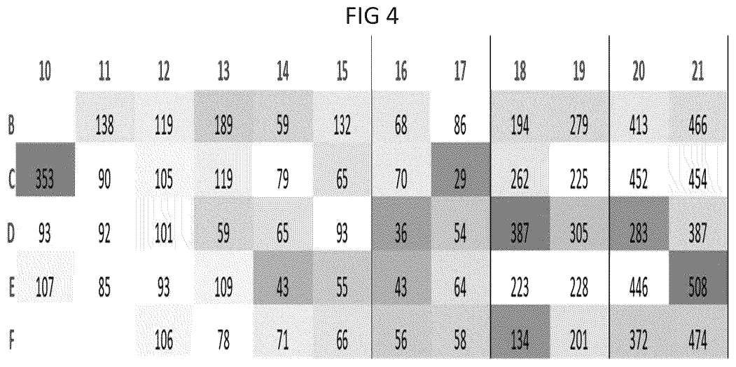

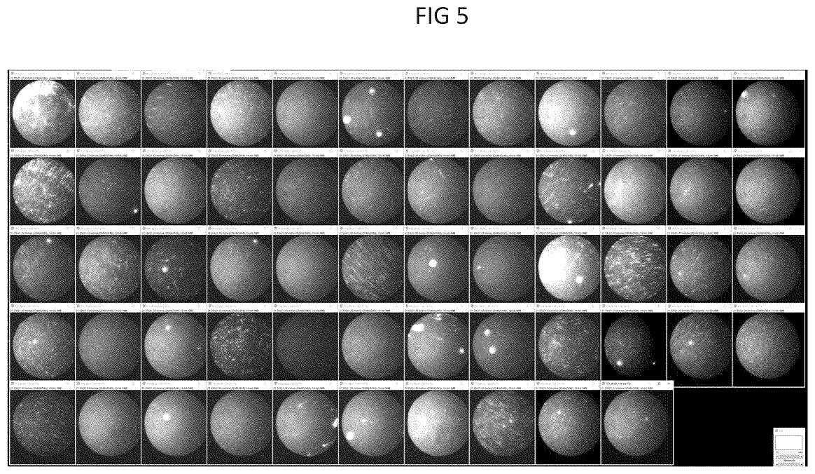

Spot counts for each of the hybridization conditions were tabulated, whereby higher counts indicated more effective hybridization buffer formulations as shown in FIG. 4.

FIG. 3 shows the surface template hybridization images (NASA results at 100 pM) of the samples corresponding to the compositions used for hybridization in Table 1. Following hybridization, RCA amplification was performed using amplification mixes with Bst (NEB). The resulting surface amplified products were again stained with intercalating DNA stains and imaged to verify hybridization specificity and effectiveness based on (FIG. 5). Hybridization conditions were evaluated based on the correlation of maximum spot counts from FIGS. 3, 4, and 5.

While preferred embodiments of the compositions and methods disclosed herein have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the methods and compositions described herein may be employed in any combination in practicing the methods and compositions of the present disclosure.

What is claimed is:

1. A method of attaching a target nucleic acid to a surface, comprising
(a) providing at least one surface bound nucleic acid that is attached to a surface; and
(b) contacting the surface bound nucleic acid to the target nucleic acid in a hybridizing composition, wherein the hybridizing composition comprises:
(i) at least one organic solvent having a dielectric constant of no greater than about 115, and
(ii) a pH buffer;
wherein the surface has a water contact angle of less than 45 degrees.

2. The method of claim 1, wherein the organic solvent is a polar aprotic solvent.

3. The method of claim 1, wherein the organic solvent is an organic solvent having a dielectric constant of no greater than 40.

4. The method of claim 1, wherein the organic solvent is acetonitrile, alcohol, or formamide.

5. The method of claim 1, wherein the organic solvent comprises at least one functionality selected from hydroxy, nitrile, lactone, sulfone, sulfite, and carbonate.

6. The method of claim 1, wherein the organic solvent is miscible with water.

7. The method of claim 1, wherein the organic solvent is present in an amount effective to denature a double stranded nucleic acid.

8. The method of claim 1, wherein the amount of the organic solvent is at least about 5% by volume based on the total volume of the formulation.

9. The method of claim 1, wherein the amount of the organic solvent is in the range of about 5% to 95% by volume based on the total volume of the formulation.

10. The method of claim 1, wherein the amount of the pH buffer is no greater than 90% by volume based on the total volume of the formulation.

11. The method of claim 1, further comprising a molecular crowding agent.

12. The method of claim 1, wherein the molecular crowding agent is selected from the group consisting of polyethylene glycol (PEG), dextran, hydroxypropyl methyl cellulose (HPMC), hydroxyethyl methyl cellulose (HEMC), hydroxybutyl methyl cellulose, hydroxypropyl cellulose, methycellulose, and hydroxyl methyl cellulose, and any combination thereof.

13. The method of claim 1, wherein the molecular crowding agent is polyethylene glycol (PEG).

14. The method of claim 1, wherein the molecular crowding agent has a molecular weight in the range of about 5 k to 40 k.

15. The method of claim 1, wherein the amount of the molecular crowding agent is at least about 5% by volume based on the total volume of the formulation.

16. The method of claim 1, wherein the amount of the molecular crowding agent is less than 50% by volume based on the total volume of the formulation.

17. The method of claim 1, further comprising an additive for controlling melting temperature of nucleic acid.

18. The method of claim 1, wherein the amount of the additive for controlling melting temperature of the nucleic acid is at least about 2% by volume based on the total volume of the formulation.

19. The method of claim 1, wherein the amount of the additive for controlling melting temperature of the nucleic acid is in the range of about 2% to 50% by volume based on the total volume of the formulation.

20. The method of claim 1, wherein the pH buffer comprises at least one buffering agent selected from the group consisting of Tris, HEPES, TAPS, Tricine, Bicine, Bis-Tris, NaOH, KOH, TES, EPPS, and MOPS.

21. The method of claim 1, wherein the pH buffer further comprises an organic solvent.

22. The method of claim 1, wherein the pH buffer comprises MOPS and methanol.

23. The method of claim 1, wherein the amount of the pH buffer is effective to maintain the pH of the formulation to be in the range of about 3 to about 10.

24. The method of claim 1, wherein the surface bound nucleic acid is attached to the surface through covalent or noncovalent bonding.

25. The method of claim 1, wherein the surface comprises one or more layers of hydrophilic polymer layers, and the surface bound nucleic acid is attached to at least one of the hydrophilic polymer layer.

26. The method of claim 1, wherein no more than 10% of the target nucleic acid is associated with the surface without hybridizing to the surface bound nucleic acid.

27. The method of claim 25, wherein the surface exhibits a level of non-specific Cy3 dye absorption of less than about 0.25 molecules/μm².

28. The method of claim 25, wherein the hydrophilic polymer coating layer comprises a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo (ethylene glycol) methyl ether methacrylate) (POEGMA), polyglutamic acid (PGA), poly-lysine, poly-glucoside, streptavidin, and dextran.

29. The method of claim 1, wherein at least 90% of the target nucleic acid anneals to the surface bound nucleic acid in less than about 15 mins.

30. The method of claim 1, wherein contacting the surface bound nucleic acid to a target nucleic acid in a hybridizing composition is performed at a temperature in the range of about 30° C. to 70° C.
